(19) United States
(12) Patent Application Publication
(10) Pub. No.: US 2026/0331309 A1
(43) Pub. Date: Oct. 22, 2026

(54) SYSTEMS AND METHODS FOR HYBRID HEALTH CARE DELIVERY

(71) Applicants: Kelly Mink, Corinth, KY (US); Benjamin Mink, Pleasant Plain, OH (US)

(72) Inventors: Kelly Mink, Corinth, KY (US); Benjamin Mink, Pleasant Plain, OH (US)

(21) Appl. No.: 19/721,019

(22) Filed: Jun. 26, 2026

Related U.S. Application Data
(63) Continuation-in-part of application No. 17/724,156, filed on Apr. 19, 2022, now Pat. No. 12,671,001.
Application No. 17/724,156 is a continuation-in-part of application No. 15/184,208, filed on Jun. 16, 2016, now abandoned.

(51) Int. Cl. G16H 80/00; G16H 50/30

(52) U.S. Cl. CPC G16H 80/00; G16H 50/30

(57) ABSTRACT

A telehealth service delivery system is disclosed for coordinating patient care activities among patients, health care providers, remote patient monitoring instruments, patient-associated devices, computing systems, and artificial intelligence resources. A session management server establishes synchronous patient health evaluation sessions among participating devices and may maintain persistent communication relationships for communicating video information, audio information, and physiological telemetry information. Operation of a synchronous session may initiate asynchronous workflows configured to perform monitoring, analysis, documentation, notifications, and other care-related activities. Information generated during asynchronous workflows may be associated with subsequent synchronous sessions to support longitudinal care. Artificial intelligence agents may participate in synchronous sessions and receive synchronized information from multiple modalities to generate clinical summaries, treatment recommendations, alerts, risk assessments, routing recommendations, and other outputs. Patient-related information may be acquired before, during, and after patient encounters to support coordinated care activities.

### I. RELATED APPLICATIONS

This application is a continuation-in-part of U.S. application Ser. No. 17/724,156, filed on Apr. 19, 2022, and now issued as U.S. Pat. No. 12,671,001 on Jun. 30, 2026, which is a continuation-in-part of U.S. application Ser. No. 15/184,208, filed on Jun. 16, 2016.

### II. FIELD OF THE INVENTION

This application discloses embodiments generally related to telehealth and remote health care delivery systems. More particularly, embodiments disclosed herein relate to systems and methods for coordinating communications, monitoring, data exchange, and care activities among patients, health care providers, remote patient monitoring instruments, patient-associated devices, mobile health (mHealth) applications, virtual check-in systems, software plugins, software extensions, software agents, application programming interfaces (APIs), computing systems, and artificial intelligence resources through synchronous and asynchronous communication architectures.

### III. BACKGROUND OF THE INVENTION

Medical services are traditionally provided to individuals at a designated health care provider facility, such as a physician's office, a physician group facility, and/or a hospital, among other variants. Typically, an individual contacts the provider when medical assistance or advice is sought. Upon request, the provider-side sets up an appointment time and date. Because of the demand for provider services, flexibility and convenience are often quickly sacrificed for immediacy. However, such scheduling often represents a poor accommodation to the patient. In addition to the inconvenience of the time and date, travel is often required (especially for rural or remotely located individuals), and wait times are unavoidable. And, because of the size and configuration of waiting rooms, transmission of infectious disease(s) is an increased concern.

For many patients, including rural residents, elderly individuals, chronically ill patients, mobility-impaired patients, and individuals requiring ongoing monitoring or specialized care, access to appropriate health care resources may involve significant travel time, scheduling constraints, and coordination among multiple providers and health care organizations. Even where physical travel is minimized, care delivery often extends beyond isolated encounters and may involve repeated interactions, follow-up activities, remote monitoring, and coordination among individual practitioners, organizational entities, caregivers, and other participants. As the availability of connected devices and communication technologies continues to expand, the amount and variety of patient-related information available before, during, and after a patient encounter likewise continues to increase. Efficiently coordinating such interactions and information while maintaining continuity of care presents continuing challenges in health care delivery.

Telehealth and telemedicine systems have commonly utilized videoconferencing and other communication technologies to facilitate synchronous consultations between patients and health care providers. Telehealth systems may also employ remote monitoring, messaging, and other mechanisms intended to improve access to care and reduce the need for physical visits. Continued and preventative care delivered through remote technologies may enhance patient outcomes, improve convenience, and reduce healthcare costs. Telehealth additionally enables consultation, diagnosis, and treatment regardless of geographic location, thereby expanding access to specialists and other health care resources.

As telehealth systems continue to evolve, patient care increasingly extends beyond isolated consultations and may involve combinations of synchronous interactions, asynchronous workflows, remote patient monitoring instruments, patient-associated devices, and coordination among multiple individual and organizational health care providers. There remains a need for telehealth delivery systems capable of supporting coordinated care activities before, during, and after patient encounters while maintaining continuity of information and communication among participating entities.

In addition, advances in connected devices, cloud computing, artificial intelligence, and distributed communication architectures have increased the amount and variety of patient-related information available during care delivery. However, integrating information generated from multiple modalities, devices, and workflows while preserving continuity across synchronous and asynchronous interactions presents continuing technical challenges. Accordingly, improved telehealth service delivery systems are desirable.

### IV. SUMMARY OF THE INVENTION

In certain embodiments, systems and methods are provided for facilitating health care delivery among patients, health care providers, remote patient monitoring instruments, patient-associated devices, and one or more computing resources. A session management system may establish one or more patient health evaluation sessions among participating devices and coordinate communication and information exchange associated therewith. Information exchanged during such sessions may include video information, audio information, physiological telemetry information, and other patient-related information. One or more health care providers may utilize such information to develop, maintain, or modify one or more treatment plans associated with a patient.

In certain embodiments, a patient health evaluation session may include persistent communication relationships among participating devices and computing resources. Such communication relationships may support continuous, periodic, intermittent, and event-driven information exchange and may facilitate synchronous interactions, asynchronous activities, or combinations thereof. Operation of a patient health evaluation session may initiate one or more workflows associated with monitoring, analysis, notifications, documentation, referrals, scheduling, and other care-related activities.

In certain embodiments, information generated during one or more interactions, workflows, and monitoring activities may be associated with subsequent patient encounters. Patient-related information may be acquired from remote patient monitoring instruments, patient-associated devices, software resources, applications, and other connected technologies before, during, and after patient interactions. Such information may be utilized to support continuity of care and coordination among participating entities.

In certain embodiments, one or more computing resources may receive and process information from multiple modalities and may generate outputs associated with documentation, recommendations, alerts, summaries, assessments, routing, and other operations. Such computing resources may cooperate with one or more health care providers and may operate during synchronous interactions, asynchronous activities, or combinations thereof.

In certain embodiments, health care delivery activities may involve combinations of individual providers, organizational providers, remote patient monitoring instruments, patient-associated devices, and computing resources participating through synchronous interactions, asynchronous activities, or combinations thereof. Information and activities associated with a patient may persist across multiple interactions and patient encounters to support coordination and continuity of care.

Additional embodiments, features, and advantages will become apparent from the following description and the appended claims. The foregoing summary is provided solely to introduce selected concepts and is not intended to identify essential features, define the scope of the claims, or limit the claimed subject matter.

### V. BRIEF DESCRIPTION OF THE DRAWING(S)

The advantages and features of the invention and the respective embodiments will become better understood with reference to the following more detailed description and claims taken in conjunction with the accompanying drawings, in which like elements are identified with like symbols, and in which:

FIG. 1 is a schematic of the system 100 described and disclosed that provides a synchronous patient evaluation session 106;

FIG. 2 is a schematic diagram illustrating an example session management architecture configured to coordinate communications among a patient device, one or more remote patient monitoring instruments, one or more provider devices, one or more artificial intelligence resources, and one or more cloud resources;

FIG. 3 is a schematic diagram illustrating an example persistent communication architecture in which one or more remote patient monitoring instruments maintain one or more persistent communication channels configured to support continuous telemetry flows and intermittent event flows;

FIG. 4 is a schematic diagram illustrating an example hybrid workflow architecture in which a synchronous patient health evaluation session may initiate one or more asynchronous workflows, one or more AI agents, one or more subsequent sessions, and one or more treatment plan updates;

FIG. 5 is a schematic diagram illustrating an example longitudinal care ecosystem associated with information generated before, during, and after a patient health evaluation session;

FIG. 6 is a schematic diagram illustrating an example AI orchestration architecture configured to process information from multiple modalities and generate one or more clinical summaries, alerts, risk assessments, and treatment plan updates;

FIG. 7 is a schematic diagram illustrating an example provider ecosystem involving individual providers, organizational providers, synchronous interactions, and coordinated treatment planning;

FIG. 8 is a schematic diagram illustrating an example coordinated treatment planning architecture in which multiple provider devices utilize patient information and physiological telemetry information to support prescription activities, referral activities, and treatment-plan maintenance activities;

FIG. 9 is a schematic diagram illustrating an example patient-associated device ecosystem comprising one or more wearable devices, smartphones, software applications, virtual check-in applications, and associated computing resources;

FIG. 10 is a schematic diagram illustrating an example continuity-of-care architecture in which information generated during one or more patient encounters contributes to longitudinal care records and subsequent encounters; and,

FIG. 11 is a schematic diagram illustrating an example AI-assisted longitudinal care architecture configured to utilize patient-associated device information and AI-assisted monitoring workflows to support continuity of care.

### VI. DETAILED DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

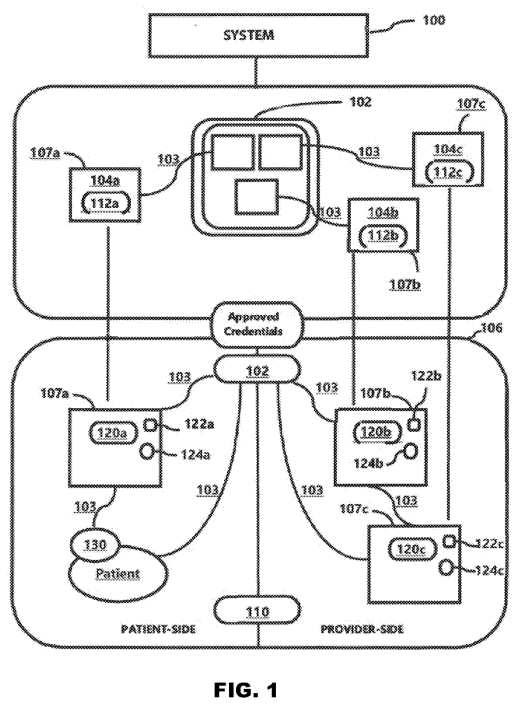

Consistent with the illustration in FIG. 1, included herewith, a telehealth delivery system 100 comprising a web portal 102 having a plurality of interfaces 104 for facilitating a synchronous patient health evaluation session 106 via an online connection 103 is described herein. The synchronous patient health evaluation session 106 may be initiated by either the patient (or patient custodian) or one of several health care providers associated with the patient and/or invited for temporary or limited association as initiated by one of the several health care providers associated with the patient.

Within the system 100, at least one of the plurality of interfaces 104 is a patient interface 104a and accommodates accessibility by a patient and/or a patient custodian, and wherein at least one of the plurality of interfaces 104 is a health care provider interface 104b. In one embodiment, the patient interface 104a includes a remote first terminal 107a and the health care provider interface 104b accommodates accessibility by two or more health care providers via a single remote second terminal 107b. In another embodiment, the patient interface 104a incudes a remote first terminal 107a and the health care provider interface 104b accommodates accessibility by two or more health care providers via a remote second terminal 107b and a remote third terminal 107c. In either embodiment, the synchronous patient health evaluation session 106 includes a coordinated maintenance or treatment plan 110 for the patient developed by the plurality of health care providers interconnected via the web portal 102. It is envisioned that as part of the plan 110 is the inclusion of the transmission of electronic dispensing of a prescription request(s) to an identified pharmacy via the web portal 102.

As used herein, the term “health care provider” is intended to be interpreted broadly and includes individual practitioners, organizations, facilities, institutions, and other entities involved in the delivery, administration, coordination, support, monitoring, diagnosis, treatment, prevention, and management of medical, behavioral, dental, pharmaceutical, rehabilitative, and wellness-related services. Health care providers may include physicians, physician assistants, nurse practitioners, registered nurses, dentists, pharmacists, psychologists, psychiatrists, therapists, counselors, social workers, physical therapists, occupational therapists, speech therapists, radiologists, pathologists, laboratory personnel, emergency medical personnel, home health providers, durable medical equipment providers, care coordinators, case managers, and other licensed, certified, accredited, or otherwise authorized personnel and entities.

As used herein, references to patient-associated devices are intended to include hardware devices and associated software resources, including mobile health (mHealth) applications, virtual check-in systems, software plugins, browser extensions, software development kits (SDKs), APIs, software agents, and combinations thereof. Such software resources may execute locally, remotely, within cloud computing environments, or combinations thereof.

In certain embodiments, one or more health care providers may be associated with National Provider Identifier (NPI) credentials or other provider identification mechanisms. Such providers may include individual providers corresponding to Type 1 National Provider Identifiers and organizational providers corresponding to Type 2 National Provider Identifiers. Type 1 providers may include physicians and other individual practitioners, while Type 2 providers may include hospitals, clinics, physician groups, pharmacies, laboratories, imaging centers, nursing facilities, ambulatory surgery centers, home health organizations, telehealth organizations, accountable care organizations, and other business entities or institutions involved in the delivery of health care services. The system 100 may utilize such identifiers for authentication, permissions, routing, scheduling, record access, billing, reimbursement, and coordination of care.

It is envisioned that one or more participating health care providers may represent departments, enterprises, provider networks, accountable care organizations, integrated delivery systems, managed care organizations, government agencies, academic institutions, research organizations, payer organizations, and other entities. Accordingly, a health care provider participating in the synchronous patient health evaluation session 106 may comprise an individual, a group of individuals, a legal entity, or combinations thereof.

In some embodiments, the health care providers described herein may further include veterinary professionals and veterinary-related entities providing care for animals. Such veterinary providers may include veterinarians, veterinary specialists, veterinary technicians, veterinary nurses, animal hospitals, veterinary clinics, animal rehabilitation providers, animal diagnostic laboratories, animal pharmacies, livestock care providers, equine practitioners, animal behavior specialists, emergency veterinary facilities, and other providers involved in companion animal, livestock, avian, aquatic, reptile, and exotic animal health. Accordingly, the patient described herein may comprise a human patient, an animal patient, or combinations thereof.

In embodiments involving veterinary care, references herein to a patient custodian may include an owner, caretaker, handler, breeder, trainer, shelter personnel, rescue personnel, agricultural personnel, and other individuals responsible for the health and well-being of an animal patient. The one or more remote patient monitoring instruments 130 may include wearable devices, implantable devices, collars, tags, harnesses, environmental sensors, activity monitors, ingestible sensors, and other monitoring instruments configured to collect physiological, behavioral, and environmental information associated with an animal patient.

Unless expressly limited by context, references herein to a health care provider are intended to encompass both individual and organizational providers, including entities associated with Type 1 and Type 2 National Provider Identifiers and analogous provider identification systems, as well as human health care providers, veterinary providers, and combinations thereof. Accordingly, the systems and methods disclosed herein are applicable to human medicine, veterinary medicine, behavioral health, dental services, animal health, and related health care disciplines.

In some embodiments, one or more AI agents 150 cooperate with the plurality of health care providers to generate, update, and maintain the coordinated maintenance or treatment plan 110 associated with the patient. The AI agents 150 may receive synchronized audio information, video information, textual information, patient records, and physiological telemetry information during the synchronous patient health evaluation session 106. The AI agents 150 may generate proposed diagnoses, treatment recommendations, medication suggestions, follow-up instructions, care pathways, and patient education materials in substantially real time. Such proposed information may be reviewed, modified, approved, or rejected by one or more health care providers participating in the synchronous patient health evaluation session 106. As additional physiological telemetry information becomes available, the AI agents 150 may dynamically update the maintenance or treatment plan 110 and generate revised patient instructions, thereby providing clinical decision support during the synchronous patient health evaluation session 106.

As used herein, the terms artificial intelligence (AI) resource and artificial intelligence (AI) agent refer to related, but not necessarily identical, concepts. In certain embodiments, an AI resource may comprise one or more computing resources, processing resources, software services, cloud-based services, machine learning models, inference engines, or combinations thereof configured to perform one or more artificial intelligence operations. An AI agent may comprise one or more software processes, executable modules, workflows, or autonomous or semi-autonomous computing entities configured to utilize one or more AI resources to perform one or more tasks described herein. Accordingly, one or more AI agents may execute using one or more AI resources, and one or more AI resources may support one or more AI agents. In some embodiments, the distinction between an AI resource and an AI agent may be logical rather than physical, and the functionality attributed to either may be distributed among one or more computing devices, servers, cloud-based environments, edge-computing resources, virtualized computing environments, containers, or combinations thereof.

The patient interface 104a accessible by the remote first terminal 107a digitally interconnected with the web portal 102 using an online connection 103, the patient interface 104a comprises one or more log-in fields 112a to evaluate the patient and/or patient custodian credentials before allowing patient and/or patient custodian access to the web portal 102. If and after patient and/or patient custodian credentials are approved the patient interface 104a launches a patient window 120a for audio/video transmission utilizing an associated camera 122a and microphone 124a.

For either of the embodiments described above concerning the health care provider interface 104b, the interface 104b comprises one or more log-in fields 112b to evaluate the first health care provider credentials and the second health care provider credentials before allowing the first health care provider and the second health care provider access to the web portal 102. If and after the first health care provider and the second health care provider credentials are approved the health care provider interface 104b launches a first health care provider window 120b and a separate second health care provider window 120c, the first health care provider window and the second health care provider window 120b, 120c providing audio/visual transmission utilizing associated camera(s) 122b, 122c and microphone(s) 124b, 124c.

The credentialing of the patient (or patient custodian) and the one or more health care providers provides the requisite security and privacy necessary to conduct a telehealth session without fear of disclosure, broadcast, and/or dissemination. In particular, it is envisioned that the credentialing of each entryway into the system 100 (via the patient-side and the health care provider-side) is consistent and in compliance with HIPAA. Via HIPAA compliance, confidence and integrity in the system 100 will be improved. HIPAA compliance also promotes secure preservation of electronic health records (EHR) and electronic medical records (EMR) as well as all such records and entries related to such records, data, and information. Initiation of the session 106 by the patient and/or patient custodian may include prompts for patient and/or patient custodian to update patient information, including non-health information and health-related information.

The patient window 120a and the at least one health care provider window 120b (and/or 120c) enable audio/video transmission between the patient and the plurality of health care providers through the web portal 102. The web portal 102 displays multiple video windows across multiple remote terminals accessed by the patient and/or patient custodian and the plurality of health care providers. Each separate remote terminal 107a, 107b, 107c comprises a two-way camera transmission for sending and receiving video signals with at least one microphone and at least one audio speaker for sending and receiving audio signals. It is envisioned that each separate remote terminal 107a, 107b, 107c may comprise one or combinations of a PC terminal, a laptop terminal, a smartphone, a smartwatch, a tablet, a television, and/or other similar technology that enables a user or users to remotely access the web portal 102.

The system 100 may include one or more remote patient monitoring instruments 130 configured for use with the patient to provide synchronous audible and/or visual biotelemetry information to the health care providers and enabling synchronous medical advice from the health care providers to the patient and/or patient custodian. The one or more remote patient monitoring instruments 130 are operably coupled and in communication through the web portal via the online interconnectivity.

It is envisioned that the one or more remote patient monitoring instruments (devices) 130 may include the following instruments and/or devices used for monitoring vital signs and/or chronic illness/disease. More particularly, vital sign remote patient monitoring instruments (devices) 130 may include such devices utilized for collecting data for blood pressure, glucose levels (e.g., glucometer), pulse oxygen data (e.g., pulse oximeter), cardio data (e.g., ECG, stethoscope), body temperature and/or body weight, as well as such devices for monitoring and controlling insulin levels and other chronic disease or illness. It is envisioned that these instruments 130 may be single-data instruments, multi-data instruments, wearable devices (e.g., wearables), and/or a combination of these items.

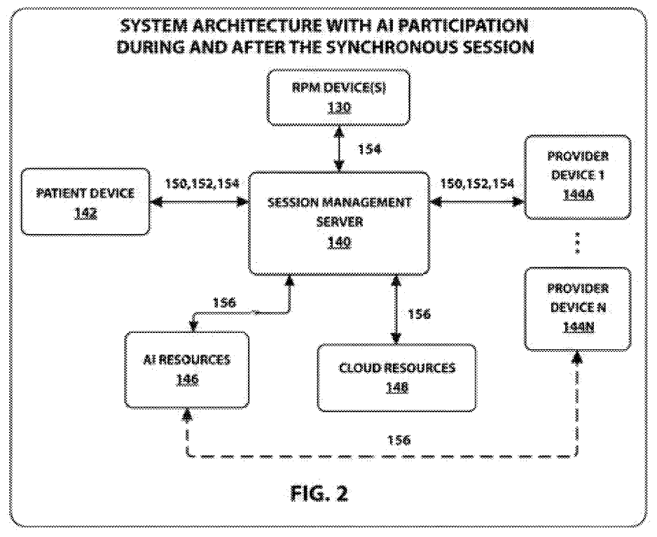

Referring now to FIG. 2, a session management server 140 may coordinate communications among a patient device 142, one or more provider devices 144A-144N, one or more remote patient monitoring instruments 130, one or more AI resources 146, and one or more cloud resources 148. In certain embodiments, information exchanged among such components may include video information streams 150, audio information streams 152, physiological telemetry information streams 154, and AI output information streams 156.

In some embodiments, the system 100 further includes a session management server 140 configured to establish, coordinate, and manage the synchronous patient health evaluation session 106 among a plurality of participating remote terminals 107a, 107b, 107c and one or more remote patient monitoring instruments 130. The session management server 140 may comprise one or more computing systems implemented in centralized, distributed, virtualized, and/or cloud-based architectures. It is envisioned that the session management server 140 may coordinate communications between patients, patient custodians, and one or more health care providers while maintaining synchronized transmission of multiple forms of data.

As used herein, a synchronous patient health evaluation session 106 is not limited to continuous contemporaneous audio and video interactions between participants. Rather, a synchronous patient health evaluation session 106 may comprise one or more persistent communication relationships maintained among remote terminals 107a, 107b, 107c, one or more remote patient monitoring instruments 130, the session management server 140, and one or more AI agents 150. Such communication relationships may be maintained through persistent network connections, streaming channels, message queues, socket connections, WebSocket connections, publish-subscribe mechanisms, event buses, and other communication pathways capable of remaining active while data transmission occurs continuously, periodically, intermittently, and/or on an event-driven basis. Accordingly, a synchronous patient health evaluation session 106 may remain established even during intervals in which no data is actively exchanged, thereby maintaining a substantially continuous communication context among participating devices and computing resources.

In certain embodiments, one or more remote patient monitoring instruments 130 maintain persistent communication channels with the session management server 140 and/or one or more AI agents 150. Such persistent communication channels may comprise WebSocket connections, bidirectional streaming channels, long-lived socket connections, and other substantially continuous communication mechanisms. Physiological telemetry information may be transmitted continuously, periodically, intermittently, or upon occurrence of predetermined events while preserving the communication relationship. Accordingly, the one or more AI agents 150 may continuously monitor the availability of incoming telemetry information and maintain awareness of patient state even during periods in which physiological measurements are transmitted only intermittently.

In some embodiments, establishment or operation of a synchronous patient health evaluation session 106 may initiate one or more asynchronous processes, workflows, and computational activities. Such asynchronous activities may continue after completion of direct interactions among patients and health care providers and may execute independently of active participation by the parties. By way of example, the session management server 140 and/or one or more AI agents 150 may initiate monitoring processes, notification processes, scheduling processes, documentation processes, analysis processes, referral processes, and other background operations associated with the patient.

In certain embodiments, the synchronous patient health evaluation session 106 may automatically spawn, instantiate, activate, and/or assign one or more AI agents 150 configured to perform asynchronous monitoring and follow-up functions. Such AI agents 150 may remain communicatively coupled to one or more remote patient monitoring instruments 130 and may continuously or intermittently monitor physiological telemetry information generated from patient devices located within a patient's home or other remote environment. The AI agents 150 may generate alerts, recommendations, summaries, treatment modifications, notifications, and requests for additional synchronous sessions responsive to detected events, threshold conditions, trends, or anomalies. Accordingly, a synchronous patient health evaluation session 106 may serve as an initiating event for persistent asynchronous care management activities extending beyond the duration of direct provider interaction.

It is envisioned that synchronous and asynchronous operations described herein may coexist within a common session architecture. Information generated during asynchronous operations may be associated with the originating synchronous patient health evaluation session 106 and subsequently utilized during later synchronous sessions. Thus, session state, patient context, AI-generated observations, physiological telemetry information, and historical information may persist across multiple synchronous and asynchronous interactions, thereby enabling longitudinal monitoring and continuity of care.

The session management server 140 may include one or more media endpoints corresponding to participating remote terminals 107a, 107b, 107c. Such media endpoints may be implemented as software processes, containers, virtual machines, and/or other executable instances configured to simultaneously process video information, audio information, and physiological telemetry information. In one embodiment, the session management server 140 utilizes a multiplexed streaming protocol configured to concurrently transmit multiple types of information through a common communication session.

The session management server 140 may further include a credential validation module configured to authenticate patient devices and health care provider devices. Authentication may include one or more of username and password validation, token-based authentication, multi-factor authentication, and other identity verification procedures. Following successful authentication, the session management server 140 registers participating devices to the synchronous patient health evaluation session 106 and assigns session identifiers and communication endpoint configurations.

In one embodiment, the session management server 140 generates a unified synchronized data stream comprising encoded video frames, audio packets, and physiological telemetry information received from the one or more remote patient monitoring instruments 130. Each component of the unified synchronized data stream may include timestamp metadata referenced to a common timing source or master clock. The synchronized data stream may utilize packetized transport protocols, including Real-Time Transport Protocol (RTP) or similar transport mechanisms, thereby preserving temporal alignment among the transmitted data components.

Video information streams 150 may comprise encoded video information. Audio information streams 152 may comprise voice or acoustic information. Physiological telemetry information streams 154 may comprise physiological measurements generated by remote patient monitoring instruments 130. AI output information streams 156 may comprise summaries, alerts, recommendations, prompts, risk scores, or combinations thereof generated by one or more AI resources 146.

Synchronization of video information, audio information, and physiological telemetry information may be achieved through one or more synchronization techniques. For example, timestamp alignment may associate telemetry measurements with corresponding video frames according to common time values. Buffering techniques may introduce controlled delays to compensate for network latency. Interpolation techniques may estimate intermediate telemetry values between measured samples. Drift correction techniques may adjust timing offsets in response to detected divergence between streams. In some embodiments, synchronization is maintained within a predetermined temporal threshold.

The session management server 140 may monitor network performance characteristics including latency, jitter, packet loss, and available bandwidth. Responsive thereto, the session management server 140 may dynamically modify encoding bitrates, frame rates, transmission priorities, and related communication parameters. During bandwidth-constrained conditions, physiological telemetry information may be assigned a transmission priority greater than video information to preserve timely delivery of clinically relevant patient information. The session management server 140 may additionally transmit session control instructions comprising timing information, layout configuration data, synchronization parameters, and rendering directives to participating remote terminals.

Each remote terminal 107a, 107b, 107c may comprise a display, camera, microphone, audio output component, network interface, and associated processing circuitry configured to receive and render synchronized media streams. The remote terminals may decode video information, audio information, and physiological telemetry information, extract timestamp metadata, and align telemetry measurements with corresponding video frames. Synchronization at the remote terminals may include buffering operations, playback timing adjustments, and latency compensation to maintain temporal consistency.

The remote terminals 107a, 107b, 107c may generate graphical user interfaces comprising one or more patient video regions, one or more health care provider video regions, and one or more telemetry display regions. Layout configurations may vary according to user role, device type, and the number of participants present during the synchronous patient health evaluation session 106. Physiological telemetry information may be displayed as numerical values, waveforms, trend graphs, visual indicators, or combinations thereof. Such telemetry information may be displayed in dedicated interface regions, overlaid upon patient video windows, and/or positioned adjacent to patient video windows.

In some embodiments, physiological telemetry information displayed within the graphical user interface remains temporally aligned with corresponding video frames. Timestamp metadata associated with telemetry information and video information may be utilized to synchronize rendering cycles such that displayed physiological measurements correspond to substantially the same time interval represented by the patient video. Dynamic updates including waveform rendering, numerical refresh operations, and graphical transitions may occur substantially in real time while preserving synchronization.

It is envisioned that the system 100 may support both real-time sessions and playback of recorded sessions. Video information, audio information, physiological telemetry information, and associated synchronization metadata may be recorded and preserved for subsequent review. During playback, timeline controls and user interactions, including scrubbing operations, may maintain synchronization between physiological telemetry information and corresponding video information. Visual indicators, color changes, and alert conditions associated with abnormal physiological measurements may likewise remain temporally aligned with the corresponding patient video.

The one or more remote patient monitoring (RPM) instruments 130 may collect physiological measurements including heart rate, blood pressure, oxygen saturation, electrocardiogram information, respiratory information, and other biotelemetry information. Such instruments 130 may generate time-stamped measurement information and communicate with a remote terminal 107a and/or directly with the session management server 140 using wired connections, short-range wireless protocols including Bluetooth communication, and/or network-based communication mechanisms.

In certain embodiments, the patient-associated digital ecosystem may further comprise mobile health (mHealth) applications, virtual check-in applications, patient portals, software plugins, browser extensions, software development kits (SDKs), application programming interfaces (APIs), software agents, and other software resources executing on smartphones, tablets, computers, wearable devices, home hubs, and other connected devices. Such software resources may communicate directly or indirectly with the session management server 140, one or more AI agents 150, and one or more health care providers.

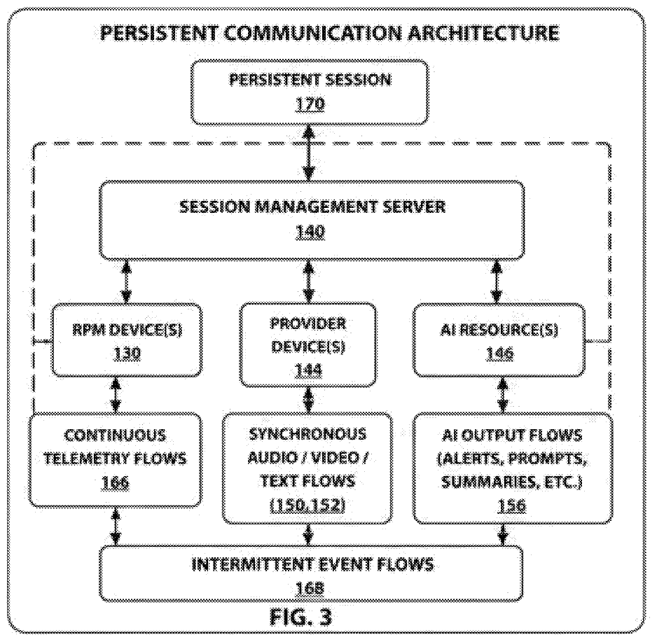

Referring to FIG. 3, remote patient monitoring instruments 130 may maintain one or more persistent communication channels 162 with the session management server 140. Such persistent communication channels may define a persistent session 170 and support continuous telemetry flows 166 and intermittent event flows 168.

Patient-associated devices may comprise smartphones, tablets, smart watches, smart rings, smart scales, smart speakers, home automation devices, medication dispensers, glucose monitoring systems, exercise equipment, sleep monitoring devices, environmental sensors, implantable devices, ingestible devices, and other smart devices. Such smart devices may execute one or more applications, plugins, software agents, and extensions configured to collect, generate, transmit, and receive patient-related information.

Virtual check-ins, electronic visits, symptom questionnaires, medication adherence activities, wellness assessments, notifications, messaging activities, and other patient interactions may occur before, during, and after a synchronous patient health evaluation session 106. Information generated during such interactions may be incorporated into synchronous sessions, asynchronous workflows, and longitudinal care activities and may be utilized by one or more AI agents 150 and participating health care providers.

In some embodiments, the asynchronous workflows comprise virtual check-ins, electronic visits, messaging interactions, symptom assessments, patient questionnaires, medication adherence evaluations, and other activities occurring without contemporaneous audio or video interaction with a health care provider. Such activities may be initiated by the session management server 140, one or more AI agents 150, or one or more participating devices and may be associated with subsequent synchronous patient health evaluation sessions.

In certain embodiments, the one or more remote patient monitoring instruments 130 may operate in conjunction with a broader patient-associated digital ecosystem comprising one or more smartphones, tablets, computers, wearable devices, applications, software agents, voice assistants, home automation devices, environmental sensors, smart appliances, medication dispensers, exercise equipment, sleep monitoring devices, smart beds, scales, implantable devices, ingestible devices, and other connected technologies configured to generate, receive, store, process, and communicate patient-related information. Such devices and technologies may communicate directly or indirectly with the session management server 140, one or more AI agents 150, one or more health care providers, and/or other participating entities.

Information generated by the patient-associated digital ecosystem may be acquired before, during, and after a synchronous patient health evaluation session 106 and may be incorporated into synchronous interactions, asynchronous workflows, and longitudinal care activities. Such information may include physiological measurements, activity information, medication adherence information, sleep information, nutritional information, environmental conditions, behavioral information, audio information, image information, location information, and other patient-related data.

In some embodiments, patient care activities are distributed across combinations of synchronous interactions and asynchronous processes involving one or more individual health care providers, organizational providers, Type 1 National Provider Identifier entities, Type 2 National Provider Identifier entities, AI agents 150, and patient-associated devices. Such activities may occur before, during, and after the synchronous patient health evaluation session 106 and may include monitoring, scheduling, referrals, documentation, treatment plan updates, notifications, communications, and coordination among participating entities.

Accordingly, the synchronous patient health evaluation session 106 need not represent an isolated event but may constitute one component of a broader hybrid care-delivery architecture extending across multiple communication sessions, asynchronous workflows, patient-associated devices, AI agents 150, individual providers, organizational providers, and longitudinal care processes. Information and actions associated with such hybrid care-delivery architectures may persist across multiple encounters and may be coordinated among a plurality of Type 1 and Type 2 National Provider Identifier participants.

In some embodiments, the one or more remote patient monitoring instruments 130 cooperate with one or more AI agents 150 configured to interpret physiological telemetry information in substantially real time. The AI agents 150 may analyze heart rate, blood pressure, oxygen saturation, electrocardiogram information, respiratory information, glucose information, body temperature information, and other physiological measurements.

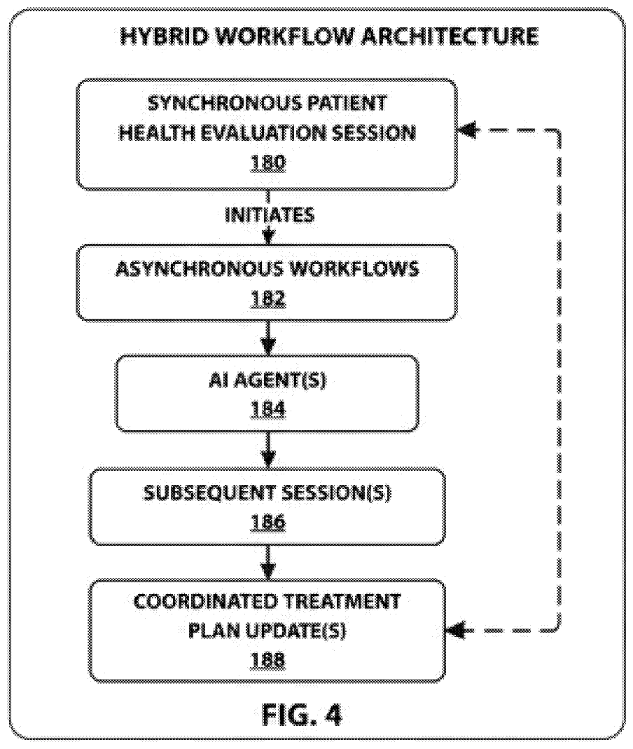

Referring to FIG. 4, operation of a synchronous patient health evaluation session 180 may initiate one or more asynchronous workflows 182. In certain embodiments, one or more AI agents 184 may participate in such workflows and may contribute to subsequent sessions 186 and coordinated treatment plan updates 188.

The AI agents 150 may generate alerts, notifications, recommendations, and visual indicators in response to detected abnormalities or trends. Such alerts may be presented to the patient, patient custodian, and participating health care providers during the synchronous patient health evaluation session 106. The AI agents 150 may further perform calibration operations, quality assessment, noise reduction, and signal validation associated with the one or more remote patient monitoring instruments 130 and may support continuous patient monitoring before, during, and after the synchronous patient health evaluation session 106.

In certain embodiments, the session management server 140 exercises centralized control over participating remote terminals 107a, 107b, 107c by transmitting session control instructions that coordinate synchronization, data distribution, and graphical interface behavior. Functionality described herein may be implemented using hardware components, software modules, executable instructions stored in memory, or combinations thereof, and may be embodied in centralized, distributed, or cloud-based computing environments. Alternative synchronization algorithms, communication protocols, and data formats may be employed without departing from the scope of the present disclosure.

In some embodiments, the system 100 further includes one or more artificial intelligence (AI) agents 150 configured to participate in the synchronous patient health evaluation session 106. The AI agents 150 may automatically join the session upon initiation thereof and receive synchronized video information, audio information, text information, and physiological telemetry information. The AI agents 150 may perform real-time speech recognition, transcription, speaker identification, extraction of structured clinical information, and generation of metadata associated with the session. Such extracted information may include symptoms, medications, allergies, diagnoses, procedures, observations, and other patient information. The one or more AI agents 150 may utilize information generated by mobile health (mHealth) applications, virtual check-ins, software plugins, APIs, software agents, and patient-associated devices to generate clinical summaries, clinician prompts, treatment recommendations, and other outputs.

The AI agents 150 may generate clinical summaries, encounter notes, problem lists, medication lists, assessment information, and other documentation during or following the synchronous patient health evaluation session 106. In some embodiments, the AI-generated information may be transmitted to electronic health record (EHR) systems or electronic medical record (EMR) systems to synchronously populate one or more patient records. The AI agents 150 may further generate prompts, reminders, recommendations, and contextual information for one or more health care providers during the session. The AI-generated documentation may be continuously updated throughout the session in response to changes in the audio information, video information, text information, and physiological telemetry information while preserving temporal relationships among such information.

In certain embodiments, the AI agents 150 operate autonomously or semi-autonomously to assist in management of the synchronous patient health evaluation session 106. The AI agents 150 may initiate actions based upon detected conditions, patient requests, provider instructions, and/or predefined clinical protocols.

The AI agents 150 may perform triage functions, recommend routing of patients to selected health care providers or specialists, request additional information from the patient, schedule follow-up sessions, initiate referrals, and generate proposed treatment recommendations. Such recommendations may be presented to one or more health care providers for review, modification, approval, or rejection.

The AI-generated outputs may be utilized by the session management server to control routing of information, initiate workflows, update session state, modify synchronization parameters, allocate computing resources, or otherwise affect operation of the telehealth service delivery system.

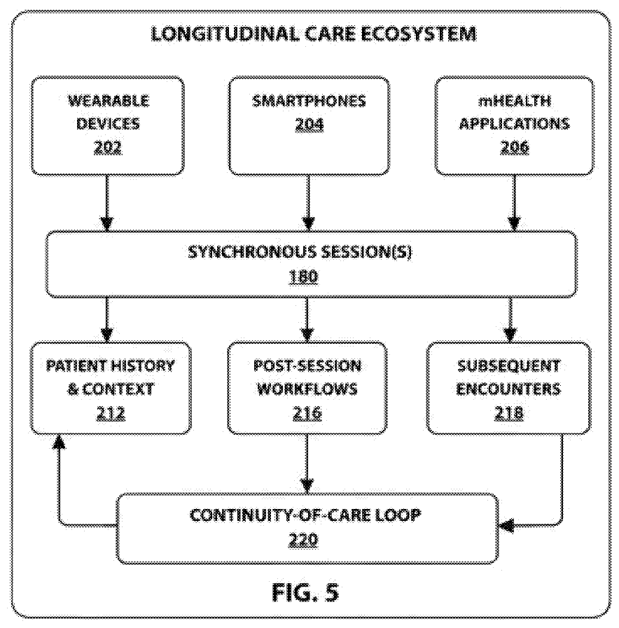

Referring to FIG. 5, patient information may be generated by wearable devices 202, smartphones 204, and mHealth applications 206. Information generated before, during, and after synchronous patient health evaluation sessions 180 may be associated with patient history information 212, post-session workflows 216, subsequent encounters 218, and continuity-of-care loops 220.

In some embodiments, the AI agents 150 may coordinate interactions among patients, patient custodians, and health care providers by controlling workflow operations and prioritizing communications based upon clinical conditions and patient-specific information.

The AI agents 150 may receive and analyze multiple synchronized modalities including audio information, video information, textual information, and physiological telemetry information. Such information may be combined through one or more multi-modal fusion models configured to identify patterns and relationships among the various data types.

The AI agents 150 may generate risk scores, anomaly indicators, severity assessments, and predictive outputs based upon simultaneous analysis of visual observations, spoken information, patient history, and physiological measurements. Abnormal conditions may be identified during the synchronous patient health evaluation session 106 and communicated to one or more participating health care providers.

By way of example, the AI agents 150 may detect respiratory distress, cardiac irregularities, neurological events, behavioral conditions, medication compliance issues, and combinations thereof through analysis of synchronized audio information, video information, textual information, and physiological telemetry information.

In certain embodiments, one or more AI agents 150 coordinate interactions among multiple health care providers participating in the synchronous patient health evaluation session 106. The AI agents 150 may manage credential verification, specialty assignments, NPI-based permissions, and participant access privileges.

The AI agents 150 may dynamically control display layouts, window positions, data presentation regions, and routing of video information, audio information, textual information, and physiological telemetry information among participating devices. Data streams may be selectively directed to individual providers based upon specialty, privileges, and clinical relevance.

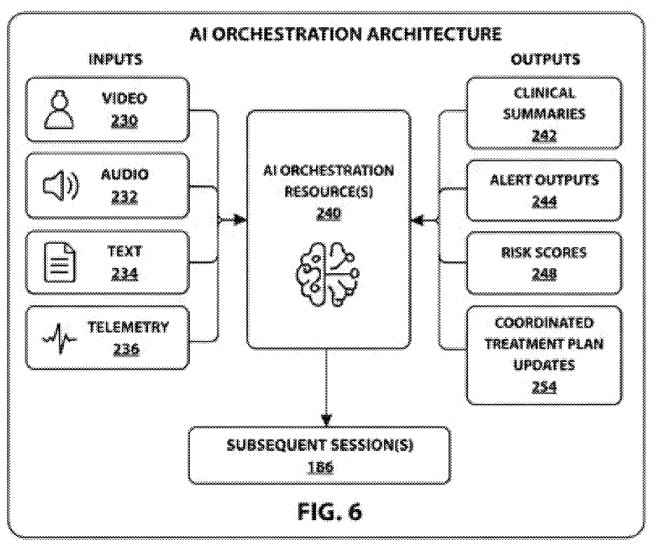

Referring to FIG. 6, video information 230, audio information 232, text information 234, and physiological telemetry information 236 may be processed by one or more AI orchestration resources 240. Such resources may generate clinical summaries 242, alert outputs 244, risk scores 248, and coordinated treatment plan updates 254 that may be utilized during subsequent sessions 186.

The AI agents 150 may additionally coordinate referrals, invitations, consultations, and collaborative treatment planning among multiple health care providers while maintaining synchronized communications among participants.

The AI agents 150 may be implemented within centralized, distributed, edge-computing, virtualized, and cloud-based environments. One or more cloud-hosted AI agents 150 may join the synchronous patient health evaluation session 106 as participants and may communicate with the session management server 140 to perform distributed inference operations and coordinated data processing.

The session management server 140 and AI agents 150 may cooperate to perform cloud-based routing, synchronization, analysis, and management of video information, audio information, textual information, and physiological telemetry information while distributing processing tasks among geographically separated computing resources.

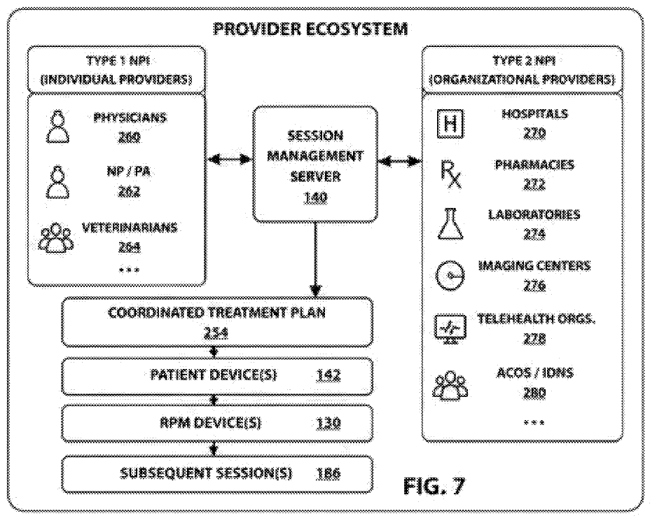

Referring to FIG. 7, synchronous interactions 284 among physicians 260, nurse practitioners 262, veterinarians 264, hospitals 270, pharmacies 272, laboratories 274, imaging centers 276, telehealth organizations 278, and accountable care organizations 280 may be coordinated through session management server 140. Information generated through such interactions may contribute to coordinated treatment plan updates 254 and may utilize patient information obtained from web portal 102 and remote patient monitoring instruments 130. Subsequent sessions 186 may facilitate continuity of care.

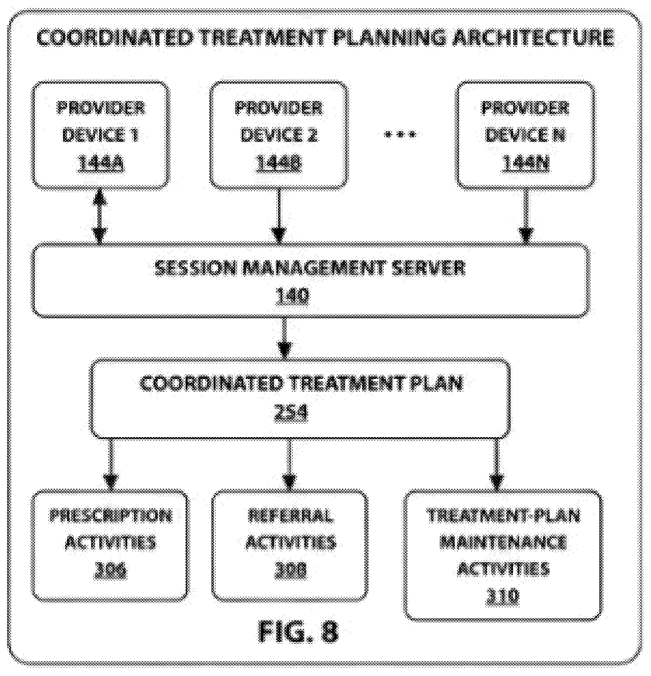

Referring to FIG. 8, provider devices 144A-144N may utilize information received through web portal 102 and remote patient monitoring instruments 130 via session management server 140 to generate coordinated treatment plan updates 254, prescription activities 306, referral activities 308, and treatment-plan maintenance activities 310.

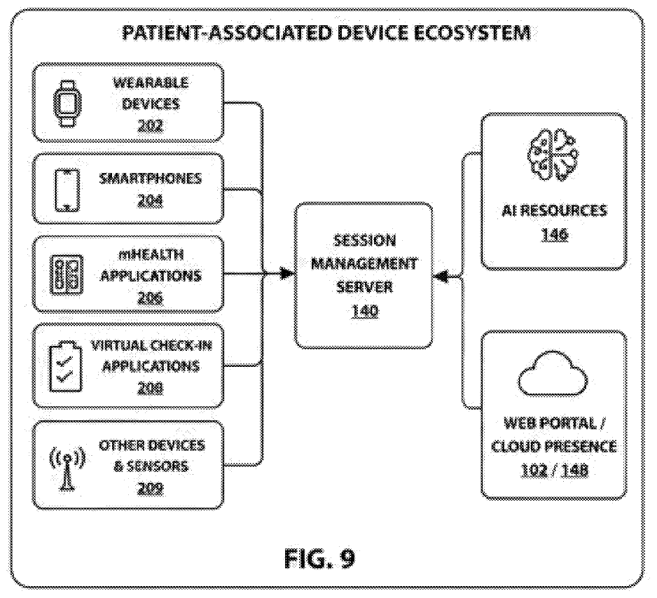

Referring to FIG. 9, a patient-associated device ecosystem may comprise wearable devices 202, smartphones 204, mHealth applications 206, virtual check-in applications 208, session management server 140, and AI resources 146 associated with web portal 102.

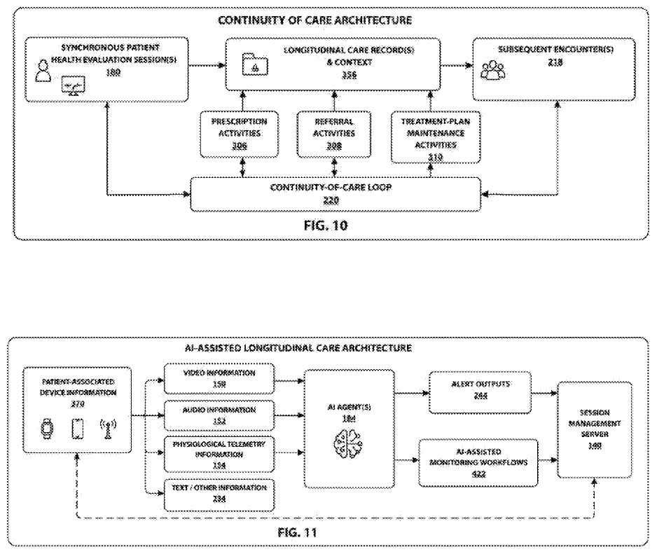

Referring to FIG. 10, synchronous patient health evaluation sessions 180 may contribute to longitudinal care records 356. Prescription activities 306 and subsequent encounters 218 may participate in continuity-of-care loops 220.

Referring to FIG. 11, patient-associated device information 370, AI output information streams 156, video information 150, audio information 152, physiological telemetry information 154, and AI agents 184 may cooperate to generate alert outputs 244 and AI-assisted monitoring workflows 422.

It is envisioned that the synchronous patient health evaluation session 106 may include advertising and/or promotion. In one embodiment, the advertisement/promotion may be included at the initiation of the session 106. In another embodiment, the advertisement/promotion may be included at the conclusion of the session 106. In another embodiment, the advertistement/promotion may be included at the initiation and conclusion of the session 106. The advertisement/promotion may assume a variety of forms, including a fixed image, fixed image with voiceover, video, video with voiceover, and/or a combination thereof.

It is also envisioned that the system 100 may be utilized in delivery of a number of telehealth services, including general health maintenance, annual exams, and/or specialized medical services. For example, including but limited to the following specialized medical services and/or areas of concern, the system 100 may include cardiology, dentistry, dermatology, endocrinology, gastroenterology, genetics, gerontology, gyneology, hematology, hepatology, immunology, infectious disease, internal medicine, nephrology, neurology, obstetrics, oncology, ophthalmology, orthopedics, otolaryngology, pallitative medicine, pathology, pediatrics, podiatry, psychiatry, pulmonology, radiology, rheumatology, serology, toxicology, urology, general and specific surgical cases, and emergency and/or disaster medicine. Other umbrella services, including athletic training and/or sports medicine, aviation, family medicine, intensive care, military medicine, rehabilitation services, rural medicine, space medicine, transplantation medicine, tropical medicine, and/or wilderness medicine, may be encompassed by the system 100.

It is envisioned that specific health care and/or medical services may be delivered through the telehealth system 100 described above. For example, these services may include providing advice and/or recommendations related to an existing or a new medical condition, diagnosing and/or treating an existing or a new medical condition, providing immediate or concurrent referral service(s) based on an existing or new medical condition, prescribing medication or other treatment measures, periodic medical monitoring of one or more existing medical condition(s), providing follow-up monitoring and/or routine examination(s) and/or check-ups, mental health counseling and/or referral, providing general health or health-related information, and/or wellness information and/or counseling.

It is envisioned that the system 100 may include entry and/or verification of credentials utilizing a variety of personal information related to the patient. For example, including but not limited to, patient name, address, contact information (e.g., cell or phone number, email address), age, gender, height, weight, medical history, current medicine(s), insurance information, medical power of attorney, allergy information, general or specific health or medical records, and/or pharmacy information. Additional information that may be used (but not necessarily required) to initiate a telehealth system 100 synchronous patient health evaluation session 106 may include request for session, patient symptom(s), specific health care request(s), and/or other related information.

It is to be understood that the embodiments and claims are not limited in its application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the U.S. Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. It is intended that the application is defined by the claims appended hereto.

What is claimed is:

1. A telehealth service delivery system, comprising:
a patient device;
a plurality of health care provider devices;
one or more remote patient monitoring instruments configured to generate physiological telemetry information associated with a patient;
a session management server configured to establish a synchronous patient health evaluation session among the patient device and the plurality of health care provider devices;
wherein the session management server is configured to coordinate synchronized transmission of video information, audio information, and physiological telemetry information among the patient device and the plurality of health care provider devices; and
wherein the plurality of health care providers are configured to utilize the video information and physiological telemetry information to generate a coordinated treatment plan for the patient.

2. The system of claim 1, wherein the session management server maintains temporal synchronization between the video information and the physiological telemetry information.

3. The system of claim 1, further comprising one or more artificial intelligence agents configured to receive synchronized video information, audio information, textual information, and physiological telemetry information associated with the synchronous patient health evaluation session.

4. The system of claim 3, wherein the one or more artificial intelligence agents generate one or more outputs selected from the group consisting of clinical summaries, clinician prompts, treatment recommendations, risk scores, anomaly indicators, and combinations thereof.

5. The system of claim 4, wherein the one or more outputs are provided during the synchronous patient health evaluation session to support generation of the coordinated treatment plan.

6. The system of claim 1, wherein one or more of the plurality of health care providers participate in one or more synchronous interactions, one or more asynchronous interactions, or combinations thereof occurring during, immediately following, or subsequent to the synchronous patient health evaluation session.

7. The system of claim 6, wherein the coordinated treatment plan is generated through a combination of synchronous interactions and asynchronous interactions among the plurality of health care providers.

8. The system of claim 1, wherein the coordinated treatment plan is generated and maintained through combinations of synchronous interactions, asynchronous follow-up activities, artificial intelligence-assisted workflows, and patient-related information generated by one or more patient-associated devices.

9. A telehealth service delivery system, comprising:
a session management server;
a plurality of participating devices comprising at least one patient device, one or more health care provider devices, and one or more remote patient monitoring instruments configured to generate physiological telemetry information associated with a patient;
wherein the session management server is configured to establish a patient health evaluation session among the plurality of participating devices;
wherein the patient health evaluation session comprises a persistent communication relationship maintained among the plurality of participating devices and the session management server;
wherein the session management server is configured to coordinate synchronized transmission of video information, audio information, and physiological telemetry information associated with the patient health evaluation session; and,
wherein operation of the patient health evaluation session initiates one or more asynchronous workflows associated with the patient.

10. The system of claim 9, wherein the one or more asynchronous workflows continue after completion of direct interactions among the patient and one or more health care providers.

11. The system of claim 9, wherein patient-related information is acquired from one or more patient-associated devices before, during, and after the patient health evaluation session.

12. The system of claim 9, wherein one or more health care providers participate through combinations of synchronous interactions and asynchronous interactions occurring during, immediately following, or subsequent to the patient health evaluation session.

13. A telehealth service delivery system, comprising:
a session management server;
a plurality of participating devices comprising at least one patient device and one or more patient-associated devices configured to generate patient-related information;
wherein the session management server is configured to establish a synchronous patient health evaluation session;
wherein operation of the synchronous patient health evaluation session initiates one or more asynchronous workflows associated with a patient; and,
wherein information generated during the one or more asynchronous workflows is associated with a subsequent synchronous patient health evaluation session.

14. The system of claim 13, wherein information generated during the one or more asynchronous workflows is utilized during the subsequent synchronous patient health evaluation session.

15. The system of claim 13, wherein continuity of care associated with the patient is maintained through combinations of synchronous interactions and asynchronous interactions among a plurality of health care providers.

16. The system of claim 13, wherein the one or more patient-associated devices comprise smartphones, wearable devices, applications, home monitoring devices, medication dispensers, environmental sensors, implantable devices, ingestible devices, or combinations thereof.

17. The system of claim 13, wherein longitudinal care activities associated with the patient are coordinated among one or more Type 1 National Provider Identifier entities and one or more Type 2 National Provider Identifier entities.

18. The system of claim 13, wherein continuity of care associated with the patient is maintained through combinations of synchronous interactions, asynchronous interactions, artificial intelligence-assisted workflows, and patient-related information generated by one or more patient-associated devices.

19. The system of claim 13, wherein one or more health care providers participate through combinations of synchronous interactions and asynchronous interactions occurring before, during, immediately following, or subsequent to the synchronous patient health evaluation session.

20. The system of claim 19, wherein information generated during asynchronous interactions among the one or more health care providers is utilized during one or more subsequent synchronous patient health evaluation sessions.
